(19) United States
(12) Patent Application Publication
(10) Pub. No.: US 2021/0262976 A1
(43) Pub. Date: Aug. 26, 2021

(54) TOP GATE THIN FILM TRANSISTOR GAS SENSOR

(71) Applicant: Sumitomo Chemical Company Limited, Tokyo (JP)

(72) Inventors: Daniel Tobjork, Cambridgeshire (GB); Nicholas Dartnell, Eltisley (GB); Christopher Newsome, St. Ives (GB)

(73) Assignee: Sumitomo Chemical Company Limited, Tokyo (JP)

(21) Appl. No.: 17/262,335

(22) PCT Filed: Jul. 23, 2019
(86) PCT No.: PCT/GB2019/052060

§ 371 Date: Jan. 22, 2021

(30) Foreign Application Priority Data
Jul. 23, 2018 (GB) 1811976.8

(51) Int. Cl. G01N 27/414; G01N 33/00; G01N 27/12; H01L 29/786

(52) U.S. Cl. CPC G01N 27/4141; H01L 29/78618; G01N 27/126; G01N 33/0047

(57) ABSTRACT

A top gate thin film transistor gas sensor for detecting or measuring a concentration of a target gas. The gas sensor is configured so that the target gas can pass through the top gate and interact with a semiconducting layer of the gas sensor. The top gate may not cover a channel of the semiconducting layer disposed beneath the top gate so that the target gas may communicate with the channel without impedance by the top gate. The top gate may be patterned with channels through which the target gas may pass through the top gate to the channel in the semiconducting layer. The top gate may be permeable to the target gas allowing passage of the target gas to the channel. A substrate on which the semiconducting layer is formed may be permeable to the target gas allowing the target gas to communicate with the channel.

### BACKGROUND

Embodiments of the present disclosure relate to gas sensors comprising top gate thin film transistors. More particularly, but not by way of limitation, some embodiments of the present disclosure relate to top gate gas sensors to detect alkenes.

Bottom gate thin film transistors have been previously used as gas sensors. For example, such use of thin film transistors as gas sensors is described in Feng et al., “Unencapsulated Air-stable Organic Field Effect Transistor by All Solution Processes for Low Power Vapor Sensing” Scientific Reports 6:20671 DOI: 10.1038/srep20671. In bottom gate thin film transistor gas sensors, a semiconductor layer at the top of the transistor is able to interact with the atmosphere and/or a gas sample. The semiconductor layer is configured to undergo an electronic interaction with a gas to be detected. The transistor comprises a gate that is disposed underneath the semiconductor layer and an electrical output from the thin film transistor is proportional to the amount/concentration of the gas.

Klug et al, “Organic field-effect transistor based sensors with sensitive gate dielectrics used for low-concentration ammonia detection” Organic Electronics 14 (2013) 500-504 discloses an organic field-effect transistor containing an ion-conducting dielectric material for detection of ammonia.

### SUMMARY

The present inventors have found that gas sensors based on bottom gate thin film transistors may often produce an inaccurate and/or noisy output as a result of interaction of the semiconductor layer with the atmosphere, contaminants at the surface of the semiconductor layer, variability in contact between the semiconductor layer and source and drain electrodes in the case of top-contact bottom gate TFT gas sensors and/or poor sensitivity of bottom gate, bottom contact TFT gas sensors. Surprisingly, the present inventors have found that these adverse effects can be mitigated/removed and that gas can be effectively detected by using a top gate thin film transistor, where the semiconductor layer is disposed underneath a top gate electrode of the transistor.

In some embodiments of the present disclosure, a top-gate thin film transistor gas sensor is provided comprising a top gate electrode that is permeable to a target gas to be detected by the sensor.

In some embodiments of the present disclosure, a top-gate thin film transistor gas sensor is provided where the top gate electrode is positioned so as not to directly cover/align with an active region/channel of the semiconductor layer, so providing the target gas an unobstructed pathway to the active region/channel.

In some embodiments, the top gate electrode is patterned, e.g., comprises fingers, comb like structures and/or the like, to provide channels, openings and/or the like that allow passage of the target gas through the top gate electrode to the semiconductor layer.

In some embodiments, a top-gate thin film transistor gas sensor is provided comprising source and drain electrodes defining a channel in the semiconductor layer. The channel defined by the source and drain electrodes comprises a channel area. In some embodiments, the gas sensor comprises a top gate electrode and a dielectric layer disposed between the semiconductor layer and the top gate electrode. In some embodiments, the top gate layer comprises a polymer. In some embodiments, the top gate electrode comprises a patterned electrode defining a conductive pattern that at least partially overlaps the channel area.

In some embodiments, a method of identifying the presence and/or concentration of a target gas in an environment is provided, the method comprising measuring a response of a top-gate thin film transistor disposed in the environment, where the top gate thin film gas sensor comprises a top gate that is permeable to the target gas and/or is arranged/patterned to provide gas communication of the target gas to a channel/active region of the semi-conductor layer. The measured response of the top gate thin film transistor may, in accordance with embodiments of the present disclosure, be used to determine presence of the target gas in the atmosphere and/or a concentration of the target gas.

In some embodiments of the present disclosure a top gate thin-film transistor gas sensor configured to sense a target gas is provided that comprises a substrate with a source electrode and a drain electrode coupled with the substrate. The substrate, source electrode and drain electrode are covered by a semiconducting material. The source and the drain electrodes are spaced apart and define a channel in the semiconducting material. The thin film transistor gas sensor may comprise a stacked arrangement with a dielectric material disposed over the semiconducting material and the top gate disposed on top of the dielectric material. In some embodiments of the present disclosure, the dielectric material and the gate electrode are permeable to the target gas, and/or the substrate is permeable to the target gas

### DESCRIPTION OF THE DRAWINGS

The disclosed technology and accompanying figures describe some implementations of the disclosed technology.

FIG. 1 is a cross-section of a top gate, bottom contact organic thin film transistor gas sensor according to some embodiments of the present disclosure;

FIG. 2A is plan view of the top gate, bottom contact organic thin film transistor gas sensor of FIG. 1 in which the gate electrode is not illustrated;

FIG. 2B is a plan view of the gate electrode of the top gate, bottom contact organic thin film transistor gas sensor of FIG. 1;

FIG. 2C is a plan view of the top gate, bottom contact organic thin film transistor gas sensor of FIG. 1;

FIG. 3 is a cross-section of a top gate, top contact organic thin film transistor gas sensor according to some embodiments of the present disclosure;

FIG. 4 is a graph of drain current vs. time for a top contact organic thin film transistor gas sensor, according to some embodiments of the present disclosure, comprising a patterned aluminium top gate electrode before, during and after exposure to 1-MCP;

FIG. 5 is a graph of resistance change vs. 1-MCP concentration for two top contact organic thin film transistor gas sensors, according to some embodiments of the present disclosure, comprising a patterned aluminium gate electrode, one of the OTFT gas sensors having gold source and drain electrodes and the other having copper source and drain electrodes;

FIG. 6 is a graph of change in current vs. time on exposure to 1-MCP for two top contact organic thin film transistor gas sensors, according to embodiments of the present disclosure having a patterned aluminium gate electrode and gold source and drain electrodes, one of the OTFTs having a thiol monolayer formed on a surface of the source and drain electrodes;

FIG. 7 is a graph of current change vs. 1-MCP concentration for a top contact organic thin film transistor gas sensor, according to some embodiments of the present disclosure, where the top gate electrode comprises an unpatterned PEDOT gate electrode and a comparative top contact organic thin film transistor having an unpatterned aluminium gate electrode;

FIG. 8 is a graph of drain current vs. time for a top contact organic thin film transistor gas sensor, according to some embodiments of the present disclosure, comprising a patterned aluminium top gate electrode before, during and after exposure to methyl hexanoate; and

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, electromagnetic, or a combination thereof. Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms. For example, while some aspect of the technology may be recited as a computer-readable medium claim, other aspects may likewise be embodied as a computer-readable medium claim, or in other forms, such as being embodied in a means-plus-function claim.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The techniques introduced here can be embodied as special-purpose hardware (e.g., circuitry), as programmable circuitry appropriately programmed with software and/or firmware, or as a combination of special-purpose and programmable circuitry. Hence, embodiments may include a machine-readable medium having stored thereon instructions which may be used to program a computer (or other electronic devices) to perform a process. The machine-readable medium may include, but is not limited to, floppy diskettes, optical disks, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other type of media/machine-readable medium suitable for storing electronic instructions. The machine-readable medium includes non-transitory medium, where non-transitory excludes propagation signals. For example, a processor can be connected to a non-transitory computer-readable medium that stores instructions for executing instructions by the processor.

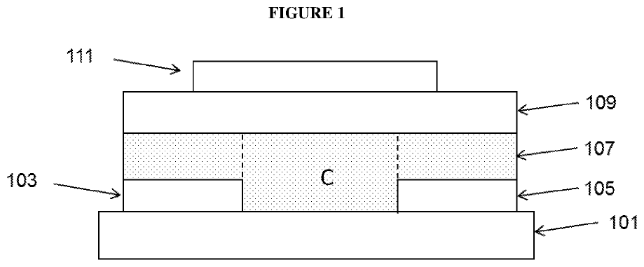

FIG. 1, is a schematic illustration of a top gate, bottom contact TFT gas sensor, in accordance with some embodiments of the present disclosure, comprising a source electrode 103 and a drain electrode 105 supported on/coupled with a substrate 101 and defining a channel C in a semiconducting layer 107. The top gate, bottom contact TFT gas sensor further comprises a gate electrode 111 and a dielectric layer 107 disposed between the gate electrode 111 and the semiconducting layer 107.

A layer “between” and/or “disposed between” two other layers, as described herein, may be in direct contact with each of the two layers or may be between or may be spaced apart from one or both of the two other layers by one or more intervening layers.

As used herein, a material “over” and/or “disposed over” a layer means that the material is in direct contact with the layer or is spaced apart therefrom by one or more intervening layers.

As used herein, a material “on” and/or “disposed on” a layer means that the material is in direct contact with the layer.

In some embodiments, the dielectric layer of the top-gate TFT gas sensor comprises a gas-permeable material, preferably an organic material, or more preferably a polymer material, which allows permeation through the dielectric layer of the gas or gases to be sensed. In some embodiments, the top-gate TFT gas sensor has a single dielectric layer between the gate electrode and the semiconducting layer. In some embodiments, the top-gate TFT gas sensor has more than one dielectric layer between the gate electrode and the semiconducting layer, each dielectric layer being permeable to the or each target gas.

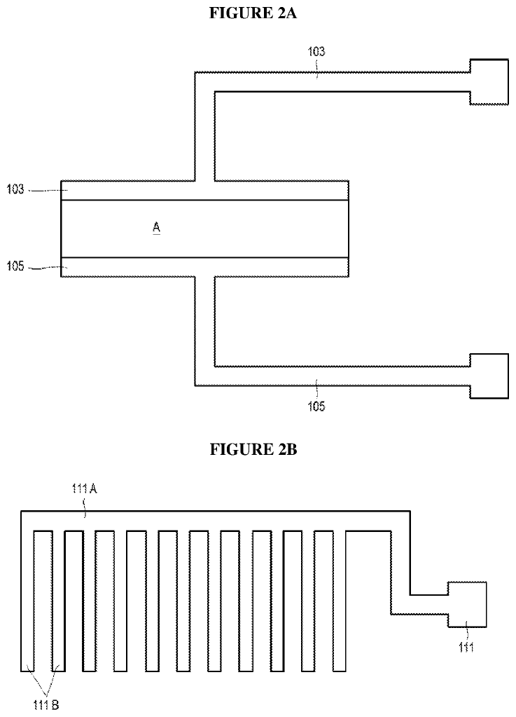

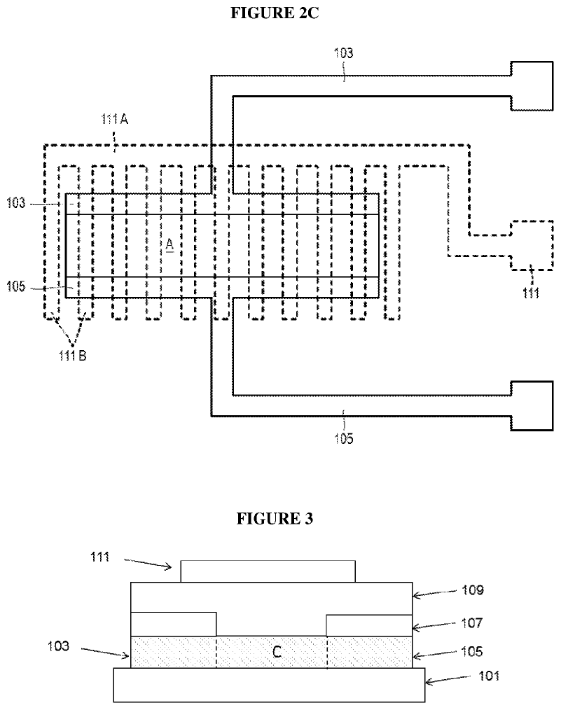

With reference to FIG. 2A, which does not show gate electrode 111 for ease of reference, the source and drain electrodes define a channel area A.

With reference to FIG. 2B, the gate electrode comprises a patterned electrode defining a conductive pattern comprising an elongate stem 111A and a plurality of fingers 111B extending from the stem, the elongate stem 111A and fingers 11B forming a conductive comb pattern. In this embodiment, the fingers extend perpendicular to the stem and are arranged parallel to one another. In other embodiments, at least some fingers are not in a parallel arrangement and/or are not perpendicular to the stem.

FIG. 2C illustrates the complete device of FIG. 1 in which the conductive pattern of the gate electrode 111 partially covers channel area A. In such embodiments, the elongate stem 111A does not overlap the channel area and at least some of the plurality of fingers 111B extending from the stem do overlap the channel area A. The gaps between fingers of the comb are void regions providing a path for one or more target gases to pass through the gate electrode. Preferably, the gaps between fingers have a greater width than the width of the fingers. Preferably, each finger has a width in the range of about 5 to 200 μm, preferably 5-150 μm.

It will be appreciated that other conductive gate electrode patterns may be provided which partially cover channel area A, thereby allowing one or more target gases to pass through a void area of the gate electrode. Exemplary structures include, without limitation, a mesh structure and a zig-zag or serpentine structure. The gate electrode defines a conductive pattern having a gate electrode area which partially overlaps the channel area, the remaining area overlapping channel area A being a void area. The void area may be a single continuous region or a plurality of discrete void regions which together form the void area

In some embodiments, a notional minimum bounding rectangle of the conductive pattern has an area which completely overlaps the channel area A. It will be appreciated that the area of the minimum bounding rectangle is made up of the conductive pattern of the gate electrode and a void area of the gate electrode.

FIG. 3 is a schematic illustration of a top gate, top contact TFT gas sensor as described in FIG. 1, except that the semiconducting layer 107 is between the substrate and the source and drain electrodes 103, 105.

In some embodiments, the top gate TFT gas sensor comprises a bottom contact TFT.

The preceding embodiments describe top gate TFTs in which the one or more target gases can permeate through the void area of a patterned gate electrode. In other embodiments, the material of the gate electrode may be permeable to the one or more target gases in which case the gate electrode may or may not be patterned. Merely by way of example, in some embodiments, the permeable gate electrodes may comprise a carbon nanotube material or a conductive polymer, for example poly(3,4-ethylenedioxythiophene) (PEDOT) doped with a polyanion such as poly(styrene sulfonate) (PEDOT:PSS) polymer or the like.

In some embodiments, the substrate may be permeable to the target gas, in which case the target gas may or may not be able to permeate through the gate electrode and/or the dielectric.

In use, a top-gate TFT gas sensor, in accordance with embodiments of the present disclosure, may be exposed to a gaseous atmosphere and connected to an apparatus, processor and/or the like for measuring a response of the gas sensor to the atmosphere resulting from interaction with/absorption by the semiconducting material and one or more gases in the atmosphere. The response may be a change in the drain current of the top-gate TFT gas sensor.

The top gate TFT gas sensor may be part of a gas sensor system comprising at least one top gate TFT gas sensor as described herein. The gas sensor system may comprise at least two different top gate TFTs as described herein. The top gate TFT gas sensors may differ in the materials/properties of their source and drain electrodes. Different responses of different TFT gas sensors in the gas sensor system may be used to differentiate between different gases in the environment. Merely by way of example, top gate TFT gas sensors with different source and drain electrodes may be used to differentiate between ethylene and 1-MCP in an environment, because of the different response to these gases by the different gas sensors.

In some embodiments of the present disclosure, the top-gate TFT gas sensor may be configured for sensing an alkene. In some embodiments of the present disclosure, the top-gate TFT gas sensor may be configured for sensing 1-methylcyclopropene (1-MCP), ethylene and/or the like. In some embodiments of the present disclosure, the top-gate TFT gas sensor may be configured to be placed in an environment in which alkenes may be present in the environmental atmosphere, for example a warehouse in which harvested climacteric fruits and/or cut flowers are stored and in which ethylene may be generated.

In some embodiments of the present disclosure, the top-gate TFT gas sensor may be configured for sensing an ester. Exemplary esters include, without limitation, esters that may be formed by the reaction of a carboxylic acid and an alkyl alcohol, such as methyl hexanoate and butyl acetate. Many esters containing small alkyl chains are fruity in smell, and are commonly used in fragrances.

In some embodiments, the top-gate TFT gas sensor may be used in a gas monitoring system. For example, the gas sensor may monitor presence of a gas and communicate with a processor to control release of the monitored gas if the monitored concentration falls to or below a threshold concentration.

The gas sensor system may be in wired or wireless communication with a controller which controls automatic release of a gas being monitored.

In some embodiments, an environment in which a gas of interest may be present may be divided into a plurality of regions. These regions may then be monitored by a plurality of the top-gate TFT gas sensors. In this way, gas concentration over a large environment, such as a warehouse or the like may be monitored, where the gas may be dispersed non-uniformly across the environment.

The gas sensor system may comprise one or more control gas sensors, optionally one or more TFT gas sensors, to provide a baseline for measurements take into account variables such as one or more of humidity, temperature, pressure, variation of sensor parameter measurements over time (such as drift due to bias stress or degradation), and gases other than a target gas or target gases in the atmosphere. One or more control gas sensors may be isolated from the atmosphere, for example by encapsulation of the or each control sensor, to provide a baseline measurement other than gases in the atmosphere.

The response of a gas sensor system as described herein to background gases other than the target gases for detection, for example air or water vapour, may be measured prior to use to allow subtraction of the background from measurements of the gas sensor when in use.

In some embodiments of the present disclosure, the source and drain electrodes may comprise any conducting material, for example a metal (e.g. gold), a metal alloy, a metal compound (e.g. indium tin oxide) and/or a conductive polymer. In some embodiments of the present disclosure, the source and drain electrodes may comprise or consist of a material capable of binding to the gas to be sensed. Merely by way of example where the gas to be detected comprises an alkene, such as 1-MCP, the source and/or drain may comprise indium tin oxide, nickel, silver, or gold. The source and drain electrodes may be a single layer of conductive material or may comprise two or more conductive layers. The source and drain electrodes may comprise a first and second layer wherein the first layer is between the second layer and the substrate. In the case of a bottom contact, top gate device the first layer may enhance adhesion of the source and drain electrodes on the substrate as compared to a single layer electrode. The first layer may be a layer of Cr. A blocking layer may be disposed on a surface of the source and drain electrodes, for example a thiol monolayer bound to the surface of the source and drain electrodes, the blocking layer being configured to prevent binding of a gas to the surface of the source and drain electrodes. Examples of suitable thiols include, without limitation, phenylthiols, alkylthiols and phenylalkylthiols wherein the benzene may be unsubstituted or substituted with one or more substituents, such as fluorinated benzenethiol s.

In some embodiments of the present disclosure, the gate electrode may be selected from any conducting material, for example a metal (e.g. aluminium), a metal alloy; a conductive metal compound (e.g. a conductive metal oxide such as indium tin oxide); or a conducting polymer, for example polyaniline or PEDOT with a charge-balancing polyanion such as PSS. In embodiments where the gate electrode is patterned, the gate preferably comprises one or more metal or metal alloy layers.

In embodiments where the gate electrode comprises an unpatterned electrode that is permeable to a target gas, the gate preferably comprises a conducting polymer.

The gate electrode may be a single layer of conductive material or may comprise two or more conductive layers. The gate electrode may comprise a first and second layer wherein the first layer is between the second layer and the gate dielectric. The first layer may enhance adhesion of the gate electrode on the gate dielectric as compared to a single layer gate. The first layer may be a layer of Cr.

In some embodiments of the present disclosure, a length of the channel (i.e. distance between the source and drain electrodes) may comprise at least 5 microns. In some embodiments of the present disclosure, the length of the channel is up to 500 microns and is preferably in the range of 5-200 microns or 5-100 microns.

Preferably, the channel length is at least 50 times, optionally at least 100 times, optionally up to 10,000 times, the thickness of the semiconducting layer. Preferably, the channel length is at least 10 times, optionally at least 50 times, optionally at least 100 times, optionally up to 10,000 times, the thickness of the dielectric layer or, if there is more than one dielectric layer, the combined thicknesses of the dielectric layers.

In some embodiments of the present disclosure, the width of the channel may be at least 100 microns, preferably at least 1 mm and may be in the range in the range of between 1-20 mm.

In some embodiments of the present disclosure, the top-gate TFT gas sensor comprises a bottom contact top-gate TFT fabricated by forming patterned source and drain electrodes followed by deposition of the semiconductor. By forming the source and drain electrodes before deposition of the semiconductor, patterning techniques may be used, for example etching, which might not be suitable for use with a top contact device due to the risk of damage the semiconductor.

In some embodiments of the present disclosure, the semiconductor material/layer may comprise of an organic semiconductor. or an inorganic semiconductor. In some embodiments of the present disclosure the semiconducting layer may comprise of a plurality of organic semiconductors.

Organic semiconductors as described herein may be selected from conjugated non-polymeric semiconductors; polymers comprising conjugated groups in a main chain or in a side group thereof; and carbon semiconductors such as graphene and carbon nanotubes.

An organic second semiconductor layer may comprise or consist of a semiconducting polymer and/or a non-polymeric organic semiconductor. The organic semiconductor layer may comprise a blend of a non-polymeric organic semiconductor and a polymer. Exemplary organic semiconductors are disclosed in WO 2016/001095, the contents of which are incorporated herein by reference.

The organic semiconducting layer may be deposited by any suitable technique, including evaporation and deposition from a solution comprising or consisting of one or more organic semiconducting materials and at least one solvent. Exemplary solvents include benzenes with one or more alkyl substituents, preferably one or more C₁₋₁₀ alkyl substituents, such as toluene, xylene and trimethylbenzene; tetralin; and chloroform.

In some embodiments of the present disclosure, the organic semiconducting layer has a thickness in the range of about 10-200 nm.

Exemplary inorganic semiconductors include, without limitation, n-doped silicon; p-doped silicon; compound semiconductors, for example III-V semiconductors such as GaAs or InGaAs; or doped or undoped metal oxides.

The, or each, dielectric layer of top gate TFT gas sensors as described herein comprises at least one dielectric material. In some embodiments of the present disclosure, the dielectric constant, k, of the dielectric material may be at least 1.0 or 1.5. In some embodiments of the present disclosure, the dielectric constant of the dielectric material is less than 100 or less than 10.

Exemplary dielectric materials are disclosed in Chem. Rev., 2010, 110 (1), pp 205-239, the contents of which are incorporated herein by reference. The dielectric material or materials may be organic, inorganic or a mixture thereof. Preferred inorganic materials include BaTiO₃, SiTiO₃, SiO₂, SiNx and spin-on-glass (SOG).

The dielectric layer preferably comprises or consists of an organic material, more preferably an aprotic polymer, for permeability of the target gas. Exemplary polymers are, polyvinylpyrrolidine (PVP), acrylates such as polymethylmethacrylate (PMMA) and benzocyclobutanes (BCBs), poly(vinyl cinnamate) P(VCn), and partially fluorinated or perfluorinated polymers, for example poly(vinylidene fluoride-co-hexafluoropropylene) P(VDF-HFP), P(VDF-TrFE-CTFE), and polymers comprising or consisting of tetrafluoroethene repeat units. The polymer may or may not be crosslinkable. Preferably, the dielectric layer is not cross-linked. In embodiments, the dielectric layer may consist of a polymer. In embodiments, the dielectric layer may be a polymer/inorganic composite, for example as described in Materials 2009, 2(4), 1697-1733, the contents of which are incorporated herein by reference. The inorganic material of the composite may be in the form of nanoparticles. The inorganic material of the composite may have a dielectric constant of at least 5, at least 10 or at least 20. In embodiments, the top-gate TFT gas sensor may comprise more than one dielectric layer, optionally a dielectric bilayer in which a first dielectric layer in direct contact with the organic semiconducting layer comprises a material having a lower dielectric constant than a material of a second dielectric layer spaced apart from the organic semiconducting layer by the first dielectric layer.

In some embodiments the, or each, dielectric layer of the top gate TFT gas sensor does not comprise a material having a protic group (hydroxyl or amine group).

In some embodiments, the, or each, dielectric layer of the top gate TFT is inert to the, or each, target gas. By “inert to the target gas” as used herein is meant that the target gas does not undergo any chemical change when brought into contact with the dielectric layer or layers of the top gate TFT gas sensor at 25° C. In some embodiments, the or each target gas is an alkene.

In some embodiments of the present disclosure, the dielectric material may be deposited by thermal evaporation, vacuum processing, lamination or from solution using, for example, spin coating or ink jet printing techniques and other solution deposition techniques discussed above.

The semiconducting layer should not be dissolved if the dielectric layer is deposited onto it from solution. Techniques to avoid such dissolution include: use of orthogonal solvents, for example use of a solvent for deposition of the dielectric layer which does not dissolve the semiconducting layer. In some embodiments, the semiconducting layer is deposited from a non-fluorinated solvent or solvent mixture and the dielectric layer is deposited from a solvent or solvent mixture containing at least one fluorinated liquid.

In some embodiments of the present disclosure, the thickness of the dielectric layer may be less than 2 micrometres, may be about 50-500 nm, and/or may be about 100-500 nm or 300-500 nm.

In some embodiments of the present disclosure, following deposition of the gate electrode over the dielectric layer, some or all dielectric material in regions that are not overlapped by the gate electrode may be removed.

The substrate of a top gate TFT gas sensor as described herein may comprise any insulating substrate, such as, for example, glass or plastic. In some embodiments of the present disclosure, the substrate may be permeable to the target gas, for example a plastic substrate.

Use of a top gate TFT gas sensor has been described herein with reference to detection of 1-MCP. This reference to 1-MCP has been made merely to show the operation of the gas sensors and is intended merely as an example of such operation as it will be appreciated by persons of skill in the art that other gases may be detected using top gate TFT gas sensors as described herein. For example any alkene which is gaseous at 20° C. and 1 atm, e.g. a C₁₋₅ alkene or the like may be detected by the top-gate TFT gas sensors in accordance with embodiments of the present disclosure. A gas sensor as described herein may be used in sensing thiols or the like.

### EXAMPLES

### General OTFT Process

A PEN substrate was baked in a vacuum oven and then UV-ozone treated for 30 s. Source and drain contacts were deposited onto the substrate by thermal evaporation of 3 nm Cr followed by 40 nm Au or Cu through shadow masks with channel length of 40, 140 or 125 μm and a channel width of 4 or 8 mm. Semiconducting Polymer 1, illustrated below, was deposited over the substrate by spin coated from a 1% w/v solution in 1,2,4-trimethylbenzene to a thickness of 20 or 40 nm and dried at 100° C. for 1 or 10 min in air. The polymer dielectric Teflon® AF2400 was spin coated from a 2.4% w/v solution in fluorinated solvent FC43 to a 300 nm thickness and dried at 80° C. for 10 min. The gate was formed by thermal evaporation of Cr (3 nm) followed by Al (200 nm) through a shadow mask to form a gate electrode having a comb structure as illustrated in FIG. 2B with comb fingers of 125 microns width and gaps of 125 microns between fingers.

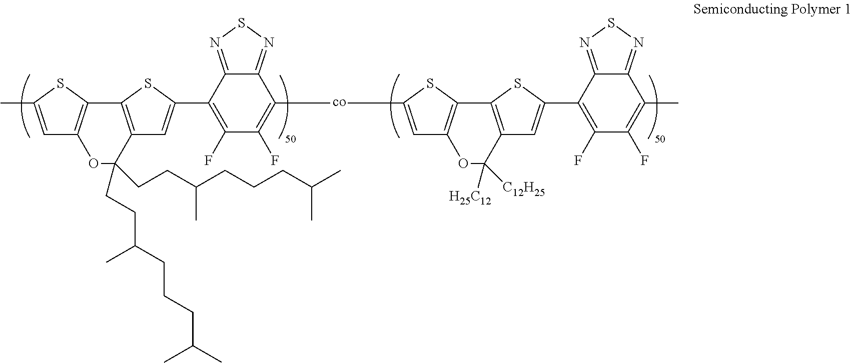

### Device Example 1

A top gate OTFT having Au source and drain electrodes, a 125 micron channel length, 4 mm channel width and a 40 nm thick semiconducting layer was prepared according to the General OTFT Process.

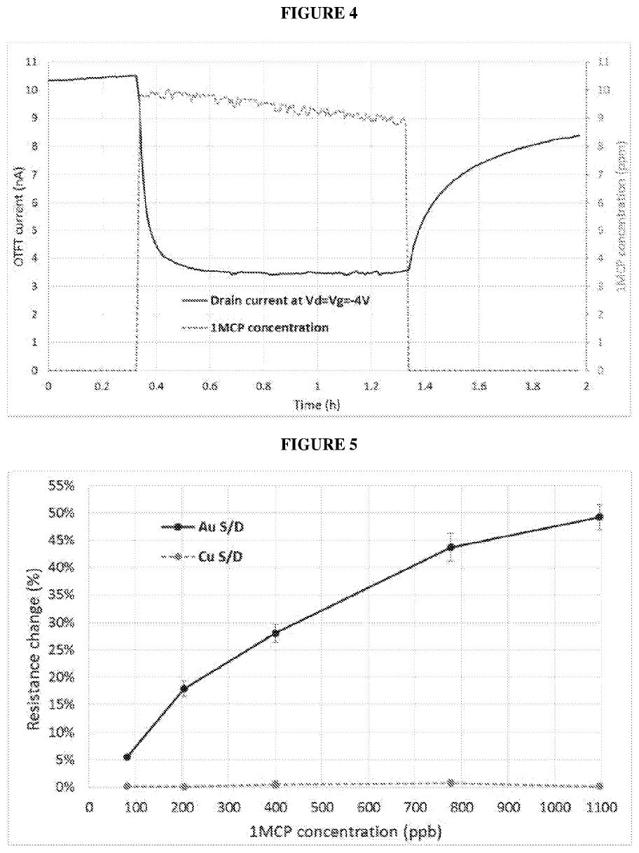

Device Example 1 was exposed to 10 ppm 1-MCP at a gas flow rate of 50 cm³/min at a voltage of Vd=Vg=−4V. With reference to FIG. 4, drain current fell by more than 60%, corresponding to an increase in resistance of more than 200%. The drain current recovered when 1-MCP was no longer present in the gas flow.

### Device Example 2

A top gate OTFT having Au source and drain electrodes, a 125 micron channel length, 8 mm channel width and a 20 nm thick semiconducting layer was prepared according to the General OTFT Process. The source and drain contacts had a width of 200 microns.

### Device Example 3

A top gate OTFT was prepared as described in Device Example 2 except that copper source and drain electrodes were used in place of gold.

Device Examples 2 and 3 were each exposed to 1-MCP for 1 hour and then left to recover in humid air free of 1-MCP for 2-6 hours between each exposure, the 1-MCP concentration being increased each hour (200, 400, 800 and 1100 ppm 1-MCP) at Vd=Vg=−4V.

With reference to FIG. 5, resistance of Device Example 2 increases with increasing 1-MCP concentration however very little or no change in resistance was observed for Device Example 3 under the same conditions. The different responses of Device Examples 2 and 3 to 1-MCP demonstrates that different OTFTs as described herein may be used to differentiate between different gases in an environment.

Without wishing to be bound by any theory, it is believed that 1-MCP binds to the gold source and drain electrodes of Device Example 2, changing the work function of the source and drain electrodes at the electrode/semiconductor interface.

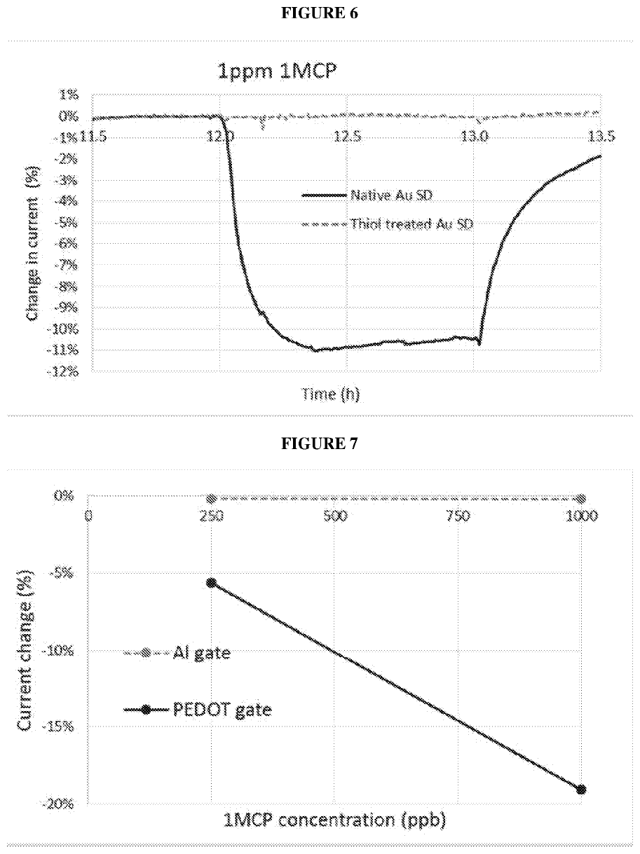

The effect of 1-MCP on the gold source and drain electrodes is shown in FIG. 6 which compares two top gate devices made according to the general OTFT process with gold source and drain electrodes, and in which a thiol monolayer was formed on the surface of the gold electrodes of one of the two devices. A reversible change in drain current was observed for the OTFT upon exposure to 1 ppm 1-MCP (at 12-13 hours as shown in FIG. 6) with untreated source and drain electrodes whereas no such change was observed for the device in which the gold surface is blocked by the thiol monolayer.

### Device Example 4

A device with a 40 micron channel length, 4 mm channel width and source/drain and 200 micron wide source and drain contacts was prepared according to the General OTFT Process except that an unpatterned gate electrode of PEDOT:PSS was formed by drop casting of PEDOT:PSS (Clevios PH1000) onto the dielectric layer to cover the whole of the channel area. For the purpose of comparison, a device was prepared according to the general device process except that the gate electrode was formed by evaporating an unpatterned layer of aluminium over the whole of the channel area.

These devices were exposed to 250 ppb and 1000 ppm 1-MCP at a drain and gate voltage of −4V. With reference to FIG. 7, a change in current is observed for Device Example 4 with a greater change being observed at the higher concentration. In contrast, no change was observed for the comparative device.

Without wishing to be bound by any theory, 1-MCP is able to permeate through the PEDOT:PSS gate of Device Example 4 but not through the aluminium gate of the comparative device.

### Device Example 5

A device was prepared according to the General OTFT Process except that the fingers were deposited through a shadow mask having 100 micron wide fingers and 200 micron gaps between fingers.

### Device Example 6

A device was prepared as described for Device Example 5 except that the shadow mask finger width was 100 microns and the gap between fingers of the shadow mask was 100 microns.

### Device Example 7

A device was prepared as described for Device Example 5 except that the shadow mask finger width was 200 microns and the gap between fingers of the shadow mask was 100 microns.

Gate electrode fingers and gaps obtained using the shadow masks of Device Examples 5-7 were measured, and sizes are set out in Table 1 (it will be appreciated that the finger width of the shadow mask as described in Device Examples 5-7 corresponds to the gap between fingers of the gate electrode, and the gap between fingers of the shadow mask corresponds to the finger width of the gate electrode).

As set out in Table 1, the gap between fingers had little effect on the resistance change upon exposure to 1 ppm of 1-MCP whereas the finger width had a significant effect on the resistance change. Without wishing to be bound by any theory, wider aluminium fingers provide less area for the 1-MCP to penetrate and laterally diffuse within the dielectric layer and semiconductor layer and reach the source and drain electrodes in the channel region where charge accumulation takes place when a gate voltage is applied.

TABLE 1

| Gate electrode finger | Gate electrode gap | Resistance change at |
|---|---|---|
| width (microns) | width (microns) | 1 ppm 1-MCP (%) |
| 191 | 108 | 2 |
| 91 | 106 | 32 |
| 95 | 204 | 34 |

### Device Example 8

A top gate OTFT was prepared as described in Device Example 1.

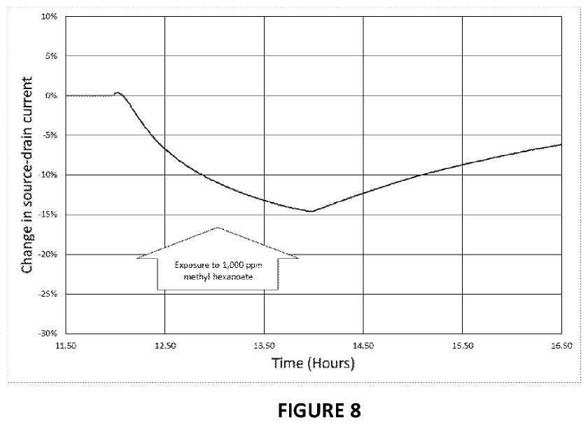

Device Example 8 was exposed to 1,000 ppm methyl-hexanoate at a gas flow rate of 50 cm³/min at a voltage of Vd=Vg=−4V. With reference to FIG. 8, drain current fell by more than 13%. The drain current recovered when methyl-hexanoate was no longer present in the gas flow.

Although the present invention has been described in terms of specific exemplary embodiments, it will be appreciated that various modifications, alterations and/or combinations of features disclosed herein will be apparent to those skilled in the art without departing from the scope of the invention as set forth in the following claims.

1. A top-gate thin film transistor gas sensor, comprising:
a substrate;
a semiconductor layer disposed on the substrate;
a dielectric layer disposed on the semiconductor layer;
a gate electrode disposed over the semiconductor layer, wherein the dielectric layer is disposed between the gate electrode and the semiconductor layer;
a source electrode and a drain electrode configured to define a channel having a channel area in the semiconducting layer;
wherein the gate electrode and the dielectric layer are configured to provide for gas communication of a gas to be sensed through the dielectric layer to the semiconductor layer; and wherein the dielectric layer comprises a polymer.

2. A top-gate thin film transistor gas sensor according to claim 1 wherein the gate electrode is a patterned electrode defining a conductive pattern which partially overlaps the channel area

3. A top-gate thin film transistor gas sensor according to claim 2 wherein a notional minimum bounding rectangle of the gate electrode overlaps the whole of the channel area.

4. A top-gate thin film transistor gas sensor according to claim 1 wherein the polymer is an aprotic polymer.

5. A top-gate thin film transistor gas sensor according to claim 1 wherein the polymer is inert to one or more target gases.

6. A top-gate thin film transistor gas sensor according to claim 5 wherein the one or more target gases are alkenes.

7. A top-gate thin film transistor gas sensor according to claim 1 wherein the dielectric layer is the only dielectric layer between the organic semiconducting layer and the gate electrode.

8. A top-gate thin film transistor gas sensor according to claim 1 wherein the gate electrode comprises an elongate stem and a plurality of laterally spaced fingers extending from the stem and overlapping the channel area.

9. A top-gate thin film transistor gas sensor according to claim 8 wherein the laterally spaced fingers each have a width of no more than 200 microns.

10. A top-gate thin film transistor gas sensor according to claim 1 wherein the source and drain electrodes comprise gold.

11. A top-gate thin film transistor gas sensor according to claim 1 wherein the dielectric layer has a thickness of 50-1000 nm.

12. A top-gate thin film transistor gas sensor according to claim 1 wherein the gate electrode comprises or consists of one or more metals.

13. A top-gate thin film transistor gas sensor according to claim 1 wherein the top-gate thin film transistor is a bottom contact, top gate thin film transistor.

14. A top-gate thin film transistor gas sensor according to claim 1 wherein the semiconductor is an organic semiconductor.

15. A top-gate thin film transistor according to claim 1 for detection of an alkene.

16. A top-gate thin film transistor gas sensor according to claim 15 wherein the alkene is 1-methylcyclopropene.

17. A top-gate thin film transistor according to claim 1 for detection of an ester.

18. A top-gate thin film transistor gas sensor according to claim 17 wherein the ester is methyl hexanoate or butyl acetate.

19. A method of identifying the presence and/or concentration of at least one target gas in an environment, the method comprising measurement of a response of a top-gate thin film transistor gas sensor according to claim 1 in the environment and determining from the measured parameter if the at least one target gas is present and/or determining a concentration of the at least one target gas.

20-22. (canceled)

23. A thin-film transistor gas sensor, comprising:
a substrate;
a semiconducting material deposited over the substrate and the source and drain electrodes;
source and drain electrodes in contact with the semiconducting material and spaced apart to define a channel;
a dielectric material deposited over the semiconducting material;
a gate electrode deposited over the dielectric material;
wherein the dielectric material and the gate electrode are permeable to the target gas, and/or the substrate is permeable to the target gas.
